(19) United States
(12) Patent Application Publication
(10) Pub. No.: US 2022/0047427 A1
(43) Pub. Date: Feb. 17, 2022

(54) Wound Covering Device

(71) Applicant: Joe Brantzeg, Blackfoot, ID (US)

(72) Inventor: Joe Brantzeg, Blackfoot, ID (US)

(21) Appl. No.: 17/153,936

(22) Filed: Jan. 21, 2021

Related U.S. Application Data
(60) Provisional application No. 63/066,255, filed on Aug. 16, 2020.

(51) Int. Cl. A61F 13/02

(52) U.S. Cl. CPC A61F 13/02; A61F 2013/00493

(57) ABSTRACT

A wound covering device for providing non-contact wound protection. The wound covering device comprises a wound cover component shaped as a curved body, wherein the curved body does not come in contact with a wound site. The wound covering device further comprises a flexible base that absorbs any water or moisture that attempts to enter a wound area during a shower or bathing. The wound covering device may further comprise a sealing gasket in addition to the flexible base. Either the flexible base or the gasket are configured to conform to an irregular body surface providing a water-tight seal. At least one self-application securing strap may be used to secure the wound covering device to the body over a wound.

### CROSS-REFERENCE TO RELATED APPLICATION

The present application claims priority to and the benefit of U.S. Provisional Application No. 63/066,255 filed on Aug. 16, 2020, which is incorporated herein by reference in its entirety.

### FIELD OF THE INVENTION

The present invention relates generally to the field of wound coverings. More specifically, the present invention relates to a wound covering for use in a shower, bath or other wet, moist or humid location to protect the wound from becoming wet and/or infected with, for example, bacterial, germs, mold or other pathogens that may be present in such environments. Accordingly, the present specification makes specific reference thereto. However, it should be appreciated that aspects of the present invention are also equally amenable with other like applications, devices and methods of manufacture.

### BACKGROUND

By way of background, wounds arise from many different causes, including accidents, surgical procedures, or other infliction of bodily harm to an individual's skin, tissue, and further layers of the epidermis or underlying tissue. People may suffer from acute wounds, such as punctures, abrasions, cuts, lacerations, burns or other skin irritations, while chronic wounds are classified as wounds that may take a month or more to fully heal.

Recent surgical and open wounds may need to be covered while showering or bathing to prevent the wound from becoming wet or infected. It can be incredibly difficult to keep the wound dry while still showering or bathing and generally maintaining proper body hygiene. People may use makeshift methods of protecting a wound, bandage, or cast such as wearing a garbage bag around the body or extremity; however, these methods can be impractical, inconvenient, uncomfortable, and ultimately ineffective in protecting the wound from water disturbance and ultimate spread of bacteria in and around the wound.

Another complication can arise if new skin forming over a healing wound is exposed to water. The new skin on the wound could become saturated and disconnected from the underlying skin, becoming macerated, which prolongs the healing process and causes additional pain. Further, if a wound is subjected directly to a strong stream or spray of water, pain, discomfort and wound damage can occur.

There are existing wound covering products on the market that seek to protect a wound from exposure to water during bathing, including products that are designed to pull on over casts on arms and legs. These inventions assume a certain minimum and maximum size requiring protection which may not meet the needs of the user. Other wound covers require a user to take extra precautions not to directly affix the cover onto the wound itself to avoid damage to the wound site when the covering is removed. These also come in a limited number of sizes which may not properly cover the wound during bathing. Additionally, users have reported that additional underlying wound dressings or sprays may be needed to ensure that the covering properly adheres but yet does not adhere to the wound site itself.

More particularly, there currently is no good way to provide comprehensive coverage for a wound located on a back or torso that is easy to apply on one's own. For instance, if a user has a wound on the middle of his or her back, he or she would likely require the assistance of another person to properly affix a wound covering or bandage in a way to cover the entire wound without damaging the wound site. Other water-proof wound protection systems recommended by medical professionals require the purchase and use of several separate materials which could include items such as gauze, cotton pads, medical tape and adhesive bandage tape to hold the various pieces together.

Therefore, there exists a long felt need in the art for an improved wound covering that prevents a wound from becoming wet during showering, bathing, or when the user is in an otherwise wet or humid environment. There is also a long felt need in the art for an improved wound covering that prevents a wound from becoming infected with bacterial, germs, mold, pathogens, and the like. Additionally, there is a long felt need in the art for an improved wound covering that does not need supplemental materials to protect the wound. Finally, there is a long felt need in the art for an improved wound covering that is relatively inexpensive to manufacture, and that is safe and easy to use.

In this manner, the improved covering of the present invention accomplishes all of the forgoing objectives, thereby providing an easy solution to adequately protect a wound. A primary feature of the present invention is a wound covering that covers a wound without direct contact to the wound that can be applied by the person with the wound. In this manner, the wound covering of the present invention accomplishes all of the forgoing objectives, and helps a user keep his or her wound dry and safe from infection while showering or bathing, and in other wet, humid or moist conditions.

### SUMMARY

The following presents a simplified summary in order to provide a basic understanding of some aspects of the disclosed innovation. This summary is not an extensive overview, and it is not intended to identify key/critical elements or to delineate the scope thereof. Its sole purpose is to present some concepts in a simplified form as a prelude to the more detailed description that is presented later.

The subject matter disclosed and claimed herein, in one embodiment thereof, comprises an improved wound covering device configured to provide non-contact protection for a wound. The wound covering device allows a user to easily apply a wound covering in a manner that does not require the assistance of another individual. The wound covering device may be adequately sized to cover large wound areas, such as, but not limited to the chest and back of a user or limbs. In addition, the wound covering device adequately covers a wound without the need for additional products to ensure proper adhesion or coverage, and also preserves the wound site from damage from the wound covering device itself upon removal or attachment.

The wound covering device comprises a wound cover component and a flexible base. The wound cover component comprises a curved top disposed between a pair of sidewalls. The curved top is typically semi-cylindrical or domed in shape to keep the wound cover component off of the wound. The wound cover component may be transparent and water-proof.

The flexible base comprises a plurality of sidewalls. The plurality of sidewalls may be rectangular, square, circular, or oval in configuration. The plurality of sidewalls each comprise a top edge and a bottom edge. The flexible base is attached to the wound cover component along a perimeter of the top edges. The flexible base may be water absorbent or water resistant to prevent the ingress of water into an interior of the wound covering device. The flexible base may be adapted so that the bottom edge conforms to and engages an irregularly shaped skin surface providing a water-tight seal.

The wound covering device may further comprise a gasket. The gasket is attachable to the bottom edge of the flexible base. The gasket is similarly adapted to and conforms to and engages an irregularly shaped skin surface providing a water-tight seal. The wound covering device may further comprise a skin securing component. The skin securing component may be an adhesive layer adhered to either the bottom edge of the flexible base, or to the gasket. The skin securing component further secures the wound covering device to the skin.

The wound covering device further comprises at least one securing component. The at least one securing component is attached to the flexible base. The at least one securing component is typically one or more straps for securing the wound covering device to the body. The straps are typically self-application straps configured to allow the user to apply the wound covering device in place over a wound without assistance.

In an additional embodiment, a wound covering device comprises a wound cover component and a flexible base. The wound cover component is a generally flexible, elastic sheet configured to stretch around a movable joint. An interior surface of the wound cover component may be covered with a non-stick coating to keep the wound cover component from sticking to the wound. The wound cover component may be transparent and water-proof.

The flexible base comprises a plurality of sidewalls. The plurality of sidewalls may be rectangular, square, circular, or oval in configuration. The plurality of sidewalls each comprise a top edge and a bottom edge. The flexible base is attached to the wound cover component along a perimeter of the top edges. The flexible base may be water absorbent or water resistant to prevent the ingress of water into an interior of the wound covering device. The flexible base may be adapted so that the bottom edge conforms to and engages an irregularly shaped skin surface providing a water-tight seal.

The wound covering device may further comprise a gasket. The gasket is attachable to the bottom edge of the flexible base. The gasket is similarly adapted to conforms to and engages an irregularly shaped skin surface providing a water-tight seal. The wound covering device may further comprise a skin securing component. The skin securing component may be an adhesive layer adhered to either the bottom edge of the flexible base, or to the gasket. The skin securing component further secures the wound covering device to the skin.

The wound covering device further comprises at least one securing component. The at least one securing component is attached to the flexible base. The at least one securing component is typically one or more straps for securing the wound covering device to the body. The straps are typically self-application straps configured to allow the user to apply the wound covering device in place over a wound without assistance.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and is intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a perspective view of one potential embodiment of the wound covering device of the present invention covering a wound site in accordance with the disclosed architecture;

FIG. 2 illustrates a perspective view of one potential embodiment of a gasket of the wound covering device of the present invention covering a wound site in accordance with the disclosed architecture

FIG. 3 illustrates a front view of one potential embodiment of the wound covering device of the present invention covering a wound site on the chest of a user and in accordance with the disclosed architecture;

FIG. 4 illustrates a side view of one potential embodiment of the wound covering device of the present invention covering a wound site on the chest of a user and in accordance with the disclosed architecture;

FIG. 5 illustrates a side view of one potential embodiment of the wound covering device of the present invention covering a wound site on the back of a user and in accordance with the disclosed architecture;

FIG. 6 illustrates a side view of one potential embodiment of the wound covering device of the present invention covering a wound site on the front of the leg of a user and in accordance with the disclosed architecture; and

FIG. 7 illustrates a side view of one potential embodiment of the wound covering device of the present invention covering a wound site on the knee area of a user and in accordance with the disclosed architecture.

### DETAILED DESCRIPTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They do not intend as an exhaustive description of the invention or do not limit the scope of the invention. Additionally, an illustrated embodiment need not have all the aspects or advantages shown. Thus, in other embodiments, any of the features described herein from different embodiments may be combined.

As noted above, there exists in the art a long felt need for an improved wound covering that allows a user to easily cover a wound site without the assistance of another individual or the use of additional products/methods. Further, there exists a long felt need in the art for an improved wound covering that can cover substantially larger or smaller portions of a user's body, that otherwise may be too large or small to cover with traditional wound covering means. In addition, a device is desired that mitigates further damage that would be caused by a covering coming in contact with a healing wound.

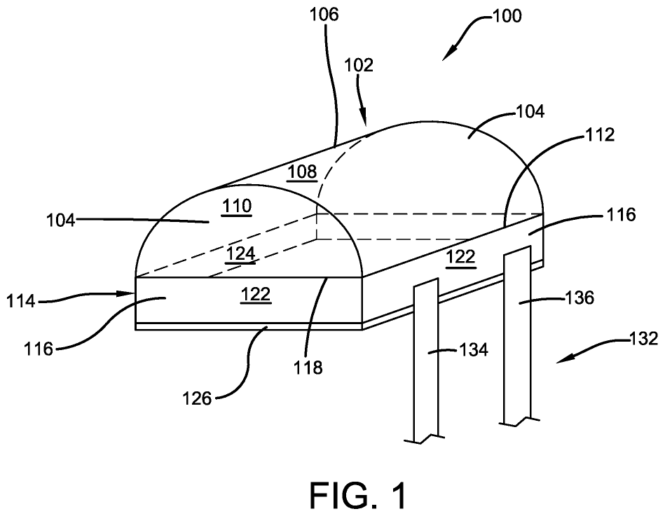

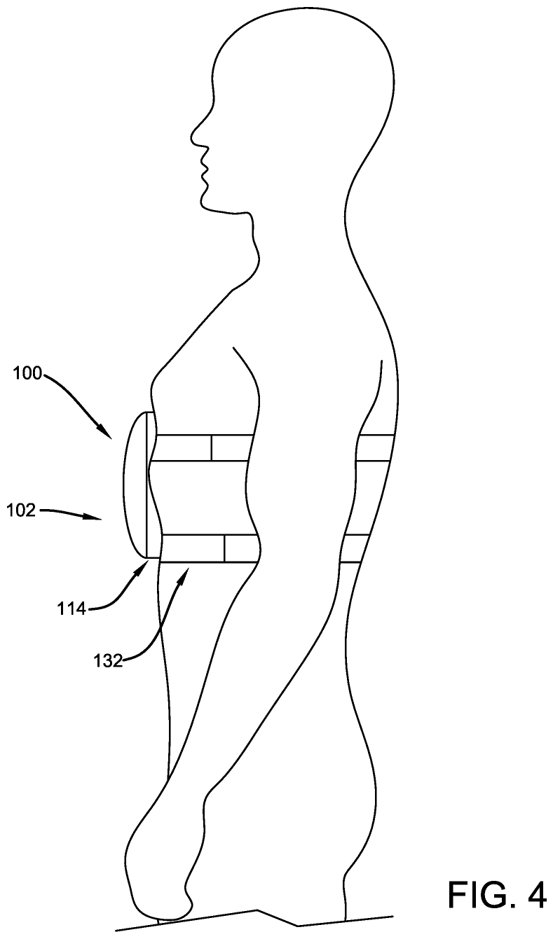

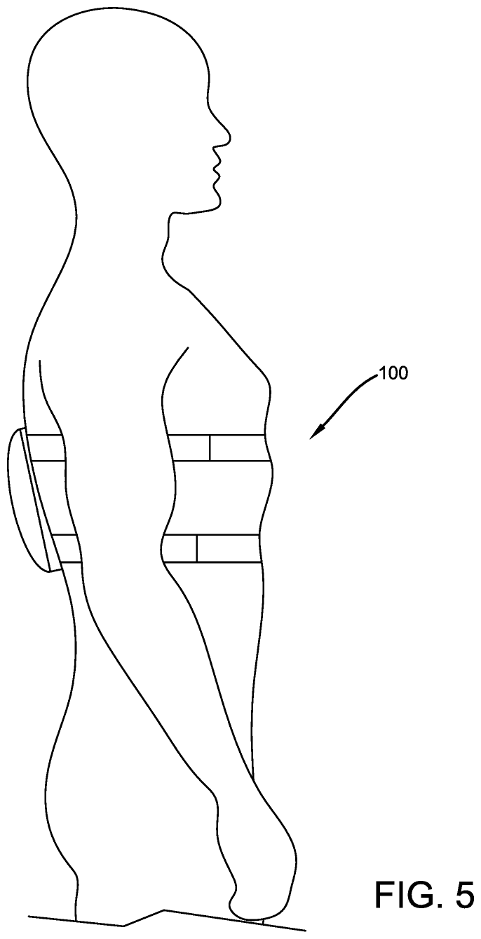

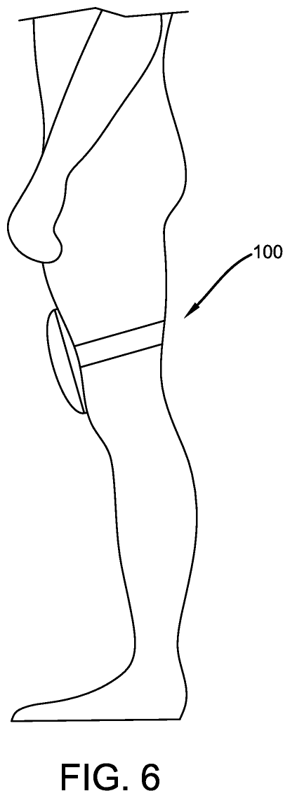

Referring initially to the drawings, FIGS. 1-6 illustrate a wound covering device 100 configured to provide non-contact protection for a wound. The wound covering device 100 is configured to allow a user to self-apply a wound covering in a manner that does not require the assistance of another individual. The wound covering device 100 is further configured to protect wounds in hard to reach places, such as on the user's back. The wound covering device 100 is water resistant to protect the wound from water during showering, bathing, or other contact with water.

The wound covering device 100 comprises a wound cover component 102 and a flexible base 114. The wound cover component 102 comprises a curved top 106. The curved top 106 may be semi-cylindrical or domed in shape to keep the wound cover component 102 from touching the wound. An exterior surface 108 faces outward and an interior surface 110 faces inward toward the wound. The interior surface 110 may be coated or covered with a non-stick coating to keep the wound cover component 102 from sticking to the wound.

When semi-cylindrical in shape, the wound cover component 102 further comprises a pair of sidewalls 104. The semi-circular shaped curved top 106 is disposed between the pair of sidewalls 104. The wound cover component 102 may be transparent and water-proof. The wound cover component 102 may be manufactured from a flexible plastic material, but may also be a semi-transparent plastic, opaque plastic, anti-fogging plastic, semi-rigid plastic, rigid plastic, or other material, and any combination thereof.

The flexible base 114 comprises a plurality of sidewalls 116. The plurality of sidewalls 116 may be rectangular, square, circular, or oval in configuration. The plurality of sidewalls 116 each comprise a top edge 118 and a bottom edge 120. An exterior surface 122 faces outward and an interior surface 124 faces inward toward the wound. The flexible base 114 is attached to the wound cover component 102 along a perimeter 112 of the top edges 118. The flexible base 114 is typically manufactured from a water absorbent or moisture wicking foam material, wherein said material absorbs any moisture or water that may otherwise enter the wound covering device 100 and saturate the wound site. Alternatively, the flexible base 114 may be water-resistant or waterproof. The flexible base 114 may be adapted so that the bottom edge 120 conforms to and engages an irregularly shaped skin surface providing a water-tight seal.

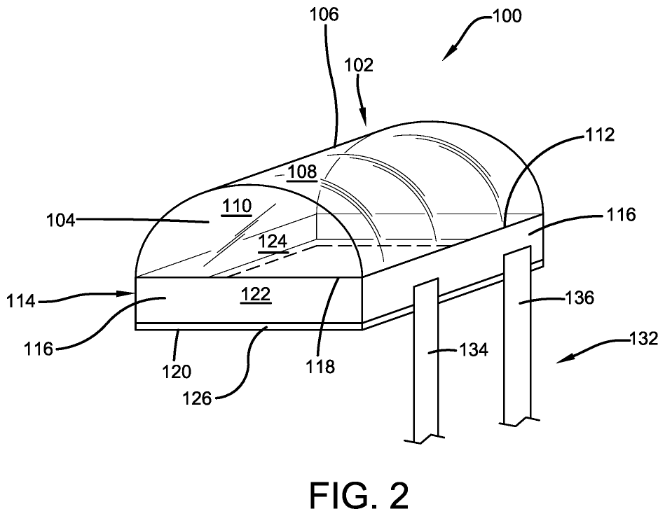

As illustrated in FIG. 2, the wound covering device 100 may further comprise a gasket 128. The gasket 128 is attachable to the bottom edge 120 of the flexible base 114. The gasket 128 is similarly adapted to conforms to and engages an irregularly shaped skin surface providing a water-tight seal. The gasket 128 may be manufactured from a flexible rubber or plastic that creates a seal via suction around the wound site.

The wound covering device 100 may further comprise a skin securing component 126. The skin securing component 126 may be an adhesive layer adhered to either the bottom edge 120 of the flexible base 114 or to the bottom of the gasket 128 where it engages the skin. The skin securing component 126 further secures the wound covering device to the skin. For example, the skin securing component 126 may be a skin-safe peel and stick adhesive surface that allow a user to stick the wound covering device 100 to the skin to further improve adhesion and sealing.

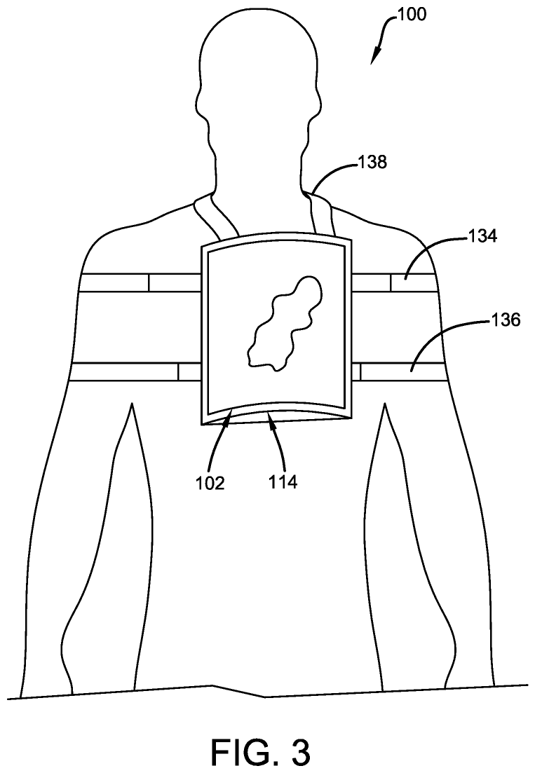

The wound covering device 100 further comprises at least one securing component 132. The at least one securing component 132 is attached to the flexible base 114. The at least one securing component 132 is typically one or more straps 134 and 136 for securing the wound covering device 100 to the body. The one or more straps 134 and 136 are typically self-application straps configured to allow the user to apply the wound covering device 100 in place over a wound without assistance. The at least one securing component 134 may further comprise a neck strap 138 as illustrated in FIG. 3. The neck strap 138 may be used to engage the user's neck to further secure the wound covering device 100 for wounds on the torso.

The one or more straps 134 and 136 allow the user to secure the wound covering device 100 against the surface of their body by placing the one or more straps 134 and 136 around their body or appendage and subsequently tightening them. As illustrated in FIGS. 3-5, the user would use the one or more straps 134 and 136 to secure the wound cover device 100 to their chest by wrapping the one or more straps 134 and 136 around their entire torso in a circumferential manner. It should be noted that the positioning of the one or more straps 134 and 136 may be chosen by the user in any orientation or position they find suitable, including positioning one of the one or more straps 134 and 136 around or near their waist.

The one or more straps 134 and 136 may be configured as a singular continuous strap that forms a single loop or a two-piece strap 130 that connects to itself. The one or more straps 134 and 136 may employ a hook and loop system to secure the one or more straps 134 and 136 in place. Further, any strap embodiment (singular or two-part construction) may be comprised of alternative constructions to affix the one or more straps 134 and 136 to themselves, such as a snap button combination, a belt hole combination, a magnetic connection, or the like. Additionally, the one or more straps 134 and 136 may employ strings which a user can use to tie and secure the wound cover device 100 around their body in the same manner. Similarly, the neck strap 138 may be a drawstring that is a continuous loop that can further serve to secure the neck around the neck or waist.

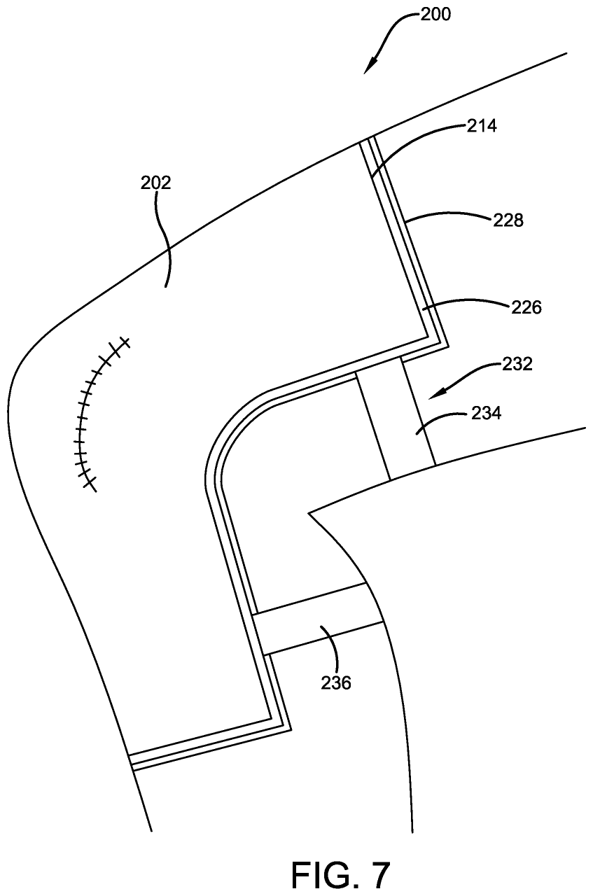

In an additional embodiment as illustrated in FIG. 7, a wound covering device 200 comprises a wound cover component 202 and a flexible base 214. The wound cover component 202 is a generally flexible, elastic sheet that can stretch around a movable joint. The wound cover component 202 does come into direct contact with a wound site. An interior surface (similar to 110) of the wound cover component may be covered with a non-stick coating to keep the wound cover component 202 from sticking to the wound. The wound cover component 202 may be transparent and water-proof.

The flexible base 214 comprises a plurality of sidewalls 216. The plurality of sidewalls 216 may be rectangular, square, circular, or oval in configuration. The plurality of sidewalls 216 each comprise a top edge (similar to 118) and a bottom edge (similar to 120). The flexible base 214 is attached to the wound cover component 202 along a perimeter (similar to 112) of the top edges (similar to 118). The flexible base 114 may be water absorbent or water resistant to prevent the ingress of water into an interior of the wound covering device 200. The flexible base 214 may be adapted so that the bottom edge conforms to and engages an irregularly shaped skin surface providing a water-tight seal.

The wound covering device 200 may further comprise a gasket 228. The gasket 228 is attachable to the bottom edge (similar to 120) of the flexible base 214. The gasket 228 is similarly adapted to conforms to and engages an irregularly shaped skin surface providing a water-tight seal. The wound covering device 200 may further comprise a skin securing component (similar to 126). The skin securing component may be an adhesive layer adhered to either the bottom edge of the flexible base 214, or to the gasket 228. The skin securing component further secures the wound covering device 200 to the skin.

The wound covering device 200 further comprises at least one securing component 232 and 234. The at least one securing component 232 and 234 is attached to the flexible base 214. The at least one securing component 232 and 234 is typically one or more straps for securing the wound covering device 200 to the body. The straps 232 and 234 are typically self-application straps configured to allow the user to apply the wound covering device 200 in place over a wound without assistance. In this embodiment, the flexible base 214 and gasket 228 are preferably extremely flexible in nature in order to be positioned in a manner consistent with the bending area of a user's body. Further, the wound cover component 202 may be coated with a non-stick film or coating which also may be comprised of a medication or medicated ointment.

Further, the wound covering devices 100 and 200 may be stored in a disposable storage container (not shown) that protects and stores the wound covering devices 100 and 200. In a differing embodiment of the wound covering devices 100 and 200, the storage container may be comprised of a non-disposable container that is further comprised of a sanitizing/decontamination means to sterilize the device after use. Further, a differing embodiment of the wound covering devices 100 and 200 may be entirely disposable (e.g. designed for a single use) or any portion thereof. As noted, each component of the wound covering devices 100 and 200 may employ an adhesive backing that would allow the user to remove that specific component and replace it with a new component of the same construction.

Notwithstanding the forgoing, the wound covering devices 100 and 200 can by any suitable size, shape, and configuration as is known in the art without affecting the overall concept of the invention, provided that it accomplishes the above stated objectives. One of ordinary skill in the art will appreciate that the shape and size of the wound covering devices 100 and 200 and its various components, as show in the FIGS. Are for illustrative purposes only, and that many other shapes and sizes of the wound covering devices 100 and 200 are well within the scope of the present disclosure. Although dimensions of the wound covering devices 100 and 200 and its components (i.e., length, width, and height) are important design parameters for good performance, the wound covering devices 100 and 200 and its various components may be any shape or size that ensures optimal performance during use and/or that suits user need and/or preference. As such, the wound covering devices 100 and 200 may be comprised of sizing/shaping that is appropriate and specific in regard to the portion of the user's body the wound covering devices 100 and 200 is designed to be.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A wound covering device for providing non-contact protection of a wound comprising:
a wound cover component comprising a pair of sidewalls and a curved top disposed between the pair of sidewalls;
a flexible base comprising a plurality of sidewalls attached to the wound cover component along a perimeter; and
at least one securing component attached to the flexible base.

2. The wound covering device of claim 1, wherein the wound covering device is water resistant.

3. The wound covering device of claim 1, wherein the curved top is semi-cylindrical in shape.

4. The wound covering device of claim 1, wherein the wound cover component is transparent.

5. The wound covering device of claim 1, wherein the flexible base is adapted to conform to a shape of an irregular surface.

6. The wound covering device of claim 1, wherein the flexible base is water absorbent.

7. The wound covering device of claim 1, wherein the flexible base is waterproof.

8. The wound covering device of claim 1 further comprising a skin securing component attached to a bottom edge of the base.

9. The wound covering device of claim 8, wherein the skin securing component is an adhesive layer.

10. A wound covering device for providing non-contact protection of a wound comprising:
a wound cover component comprising a curved top;
a flexible base comprising a plurality of sidewalls attached to the wound cover component along a perimeter;
a gasket attached to a bottom edge of the flexible base; and
at least one securing component attached to the flexible base.

11. The wound covering device of claim 10, wherein the curved top is semi-cylindrical in shape.

12. The wound covering device of claim 10, wherein the curved top is domed in shape.

13. The wound covering device of claim 10, wherein the at least one securing component is a self-application strap.

14. The wound covering device of claim 10, wherein the at least one securing component is a pair of self-application straps.

15. The wound covering device of claim 10, wherein the gasket is adapted to conform to a shape of an irregular surface.

16. The wound covering device of claim 10 further comprising a skin securing component attached to a bottom edge of the gasket.

17. The wound covering device of claim 16, wherein the skin securing component is an adhesive layer.

18. A wound covering device for protection of a wound comprising:
a wound cover component;
a flexible base comprising a plurality of sidewalls attached to the wound cover component along a perimeter;
a gasket attached to a bottom edge of the flexible base; and
at least one securing component attached to the flexible base.

19. The wound covering device of claim 18, wherein the wound cover component is a flexible, stretchable sheet.

20. The wound covering device of claim 18, wherein an interior surface of the wound cover component is covered with a non-stick coating.
